(19) United States
(12) Patent Application Publication
(10) Pub. No.: US 2016/0338651 A1
(43) Pub. Date: Nov. 24, 2016

(54) SYSTEM AND A METHOD FOR DETERMINING RISK ASSOCIATED WITH LUMBAR INTERVERTEBRAL DISC PROLAPSE

(71) Applicant: HCL Technologies Limited, Uttar Pradesh (IN)

(72) Inventors: Banumathi Palanichamy, Tamil Nadu (IN); Selvaraj Thangaraj, Tamil Nadu (IN); Shyam Thangaraju, Tamil Nadu (IN)

(21) Appl. No.: 15/075,600

(22) Filed: Mar. 21, 2016

(30) Foreign Application Priority Data
May 22, 2015 (IN) 1455/DEL/2015

(51) Int. Cl. A61B 5/00; A61B 5/0488; A61B 5/11

(52) U.S. Cl. CPC A61B 5/7275; A61B 5/4566; A61B 5/1118; A61B 5/0488; A61B 5/746; A61B 5/6801; A61B 5/0022; A61B 2090/3735

(57) ABSTRACT

The present disclosure discloses device system 102 and a method for determining risk associated with lumbar intervertebral disc prolapse. The creating module 116 may create a risk-profile corresponding to each lumbar intervertebral disc of a user. The risk-profile created may comprise annulus fibrosus related data and nucleus pulposusrelated data. The receiving module 118 may receive sensor-data corresponding to one or more muscle groups of user's interest and a lumbar region of the user. The lumbar region comprises L1 to L5 lumbar vertebrae and their adjoining IV discs. Further, the sensor-data indicates an effect of user's activity on the lumbar region of the user. The processing module 120 may process the risk-profile and the sensor-data based on a predefined value in order to determine information pertaining to a risk associated with the lumbar intervertebral disc prolapse. Further, the information may be transmitted by the transmitting module 122 to a wearable device 104 of the user.

### CROSS-REFERENCE TO RELATED APPLICATIONS AND PRIORITY

The present application claims benefit from Indian Complete Patent Application Number 1455/DEL/2015, filed on 22 May 2015, the entirety of which is hereby incorporated by reference.

### TECHNICAL FIELD

The present subject matter described herein, in general, relates to a system and method for determining risk associated with lumbar intervertebral disc prolapse.

### BACKGROUND

Lumbar intervertebral disc prolapse is a spinal condition that causes back pain as well as muscle weakness in lower part of a human-body. In general, the intervertebral disc lies between adjacent vertebrae in a vertebral column of the human-body. The intervertebral disc acts as a flexible, fibrous, compressible, connecting layer to hold the vertebrae in the Lumbar region. Further, each intervertebral disc consists of an outer fibrous ring (annulus fibrosus) which surrounds a gel-like inner core (nucleus pulposus). This outer fibrous ring composed of ligament fibers that encase the inner core. The disc prolapse condition which is also called as slipped disc or a herniated disc occurs when the outer fibrous ring of the intervertebral disc splits, resulting in the gel-like inner core bulging out of the intervertebral disc.

This causes severe back pain or neck pain in the human-body. The major cause of such problems is inactivity, poor posture of the human at work and home, and aging. People do not realize that their skeletal system mainly the spine is stressed due to inappropriate posture and lifting heavy weights under suboptimal posture. There are some exercises recommended for avoiding such slipped disc problems. But, it has been observed that the people don't take these recommendations seriously. They keep on neglecting or ignoring such problems until it becomes a serious issue. Hence, the lumbar region of the spine is particularly prone to injury especially when the spine from the hip above is used as a cantilever in when the upper body is bent forward to lift heavy weights.

### SUMMARY

This summary is provided to introduce aspects related to a system and method for determining risk associated with lumbar intervertebral disc prolapse are further described below in the detailed description. This summary is not intended to identify essential features of subject matter nor is it intended for use in determining or limiting the scope of the subject matter.

In one implementation, a system for determining risk associated with lumbar intervertebral disc prolapse is disclosed. The system comprises a processor, an input/output (I/O) interface, a memory, a creating module, a receiving module, a processing module, and a transmitting module. The creating module may create a risk-profile corresponding to each lumbar intervertebral disc of a user. The risk-profile may comprise annulus fibrosus related data and nucleus pulposusrelated data. Further, the receiving module may receive sensor-data corresponding to one or more muscle groups around the lumbar vertebral region of the user. Further, the lumbar vertebral region comprises of L1 to L5 lumbar and their adjoining IV discs. Further, the sensor-data may indicate an effect of user's activity on the lumbar region of the user. Further, the processing module may process the risk-profile and the sensor-data based on a predefined value in order to determine information pertaining to a risk associated with the lumbar intervertebral disc prolapse. Further, the transmitting module may transmit the information pertaining to the risk to a wearable device worn by the user.

In another implementation, a method determining risk associated with lumbar intervertebral disc prolapse is disclosed. The method may comprise creating, by a processor, a risk-profile corresponding to each lumbar intervertebral disc of a user. Further, the risk-profile may comprise annulus fibrosus related data and nucleus pulposusrelated data. The method may further comprise a step of receiving, by the processor, sensor-data corresponding to one or more muscle groups of user's interest and a lumbar region of the user. Further, the lumbar region may comprise L1 to L5 lumbar vertebrae. Further, the sensor-data may indicate an effect of user's activity on the lumbar region of the user. The method may further comprise a step of processing, by the processor, the risk-profile and the sensor-data based on a predefined value in order to determine information pertaining to a risk associated with the lumbar intervertebral disc prolapse. Further, the information pertaining to the risk is transmitted to a wearable device worn by the user.

In yet another implementation, a non-transitory computer readable medium embodying a program executable in a computing device for determining risk associated with lumbar intervertebral disc prolapse is disclosed. The program may comprise a program code for creating a risk-profile corresponding to each lumbar intervertebral disc of a user. Further, the risk-profile may comprise annulus fibrosus related data and nucleus pulposusrelated data. The program may further comprise a program code for receiving sensor-data corresponding to one or more muscle groups of user's interest and a lumbar region of the user. The lumbar region may comprise L1 to L5 lumbar vertebrae. Further, the sensor-data may indicate an effect of user's activity on the lumber region of the user. Further, the program may comprise a program code for processing the risk-profile and the sensor-data based on a predefined value in order to determine information pertaining to a risk associated with the lumbar intervertebral disc prolapse.

### BRIEF DESCRIPTION OF THE DRAWINGS

The detailed description is described with reference to the accompanying figures. In the figures, the left-most digit(s) of a reference number identifies the figure in which the reference number first appears. The same numbers are used throughout the drawings to refer like features and components.

FIG. 1 illustrates a network implementation illustrating communication between system and wearable device comprising sensors for determining risk associated with lumbar intervertebral disc prolapse, in accordance with an embodiment of the present disclosure.

FIG. 2 illustrates a method for determining risk associated with lumbar intervertebral disc prolapse, in accordance with an embodiment of the present disclosure.

### DETAILED DESCRIPTION

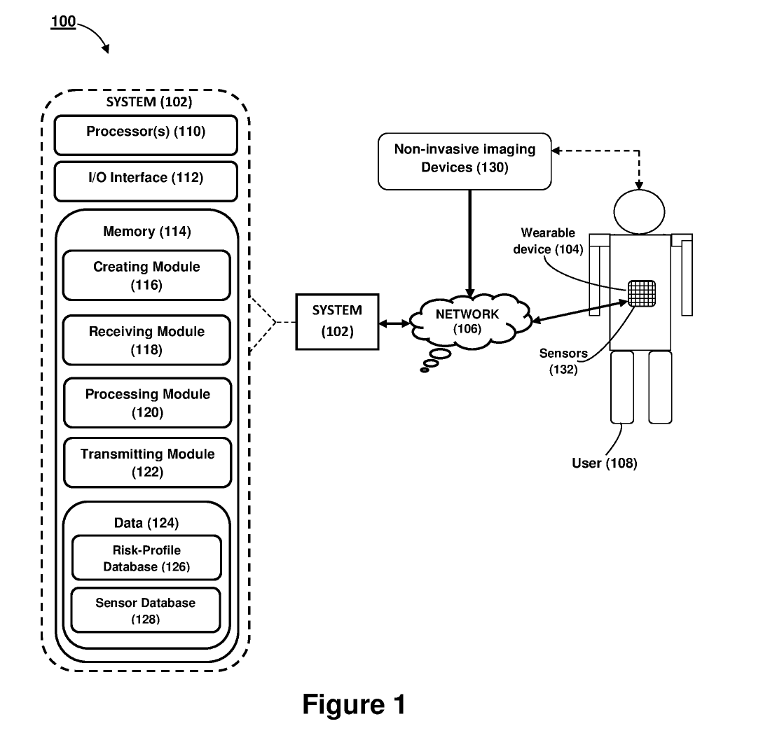

Referring to FIG. 1, a network implementation 100 of a system 102, a wearable device 104 comprising one or more sensors 132, and a non-invasive imaging device 130, for determining risk associated with lumbar intervertebral disc prolapse is illustrated, in accordance with an embodiment of the present subject matter. Although the present subject matter is explained considering that the system 102 is implemented on a server, it may be understood that the system 102 may also be implemented in a variety of computing systems, such as a laptop computer, a desktop computer, a notebook, a workstation, a mainframe computer, a server, a network server, a tablet, a mobile phone, and the like. Further, the wearable device 104 may be worn on various parts of the body of a user 108. According to embodiments of present disclosure, the system 102 may be communicatively coupled to the wearable device 104 through a network 106.

In one implementation, the network 106 may be a wireless network, a wired network or a combination thereof. The network 106 can be implemented as one of the different types of networks, such as intranet, local area network (LAN), wide area network (WAN), the internet, and the like. The network 106 may either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further the network 106 may include a variety of network devices, including routers, bridges, servers, computing devices, storage devices, and the like.

The system 102 illustrated in the FIG. 1 may further comprise a processor 110, an input/output (I/O) interface 112, and a memory 114 comprising plurality of modules, and data 124. The processor 110 may be implemented as one or more microprocessors, microcomputers, microcontrollers, digital signal processors, central processing units, state machines, logic circuitries, and/or any devices that manipulate signals based on operational instructions. Among other capabilities, the at least one processor 110 is configured to fetch and execute computer-readable instructions or modules stored in the memory 114.

The I/O interface 112 may include a variety of software and hardware interfaces, for example, a web interface, a graphical user interface, and the like. The I/O interface 112 may allow the system 102 to interact with the wearable device 104. Further, the I/O interface 112 may enable the system 102 to communicate with other computing devices, such as web servers and external data servers (not shown). The I/O interface 112 can facilitate multiple communications within a wide variety of networks and protocol types, including wired networks, for example, LAN, cable, etc., and wireless networks, such as WLAN, cellular, or satellite. The I/O interface 112 may include one or more ports for connecting a number of devices to one another or to another server.

The memory 114 may include any computer-readable medium and computer program product known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes. The memory 114 may include modules which may perform particular tasks or implement particular abstract data types.

The modules include routines, programs, objects, components, data structures, etc., which perform particular tasks or implement particular abstract data types. In one implementation, the modules may include a creating module 116, a receiving module 118, a processing module 120, and transmitting module 122. Further, the data 124 comprises risk-profile database 126, and sensor database 128.

According to embodiments of present disclosure, the system 102 and method for determining risk associated with lumbar intervertebral disc prolapse are described in detail. The human vertebral column is made up of many vertebrae which are divided into cervical (neck region), thoracic (back of chest), lumbar (lower back till the gluteal region) sacral and coccygeal (below the gluteal region) vertebrae. The vertebrae are held together by structures called inter vertebral discs which are present in between two vertebrae. This intervertebral disc is a circular structure consisting of an outer annulus fibrosus which have multiple fibrous concentric layers surrounding a jellyish inner structure called the nucleus pulposus.

The fibrous concentric layers of the annulus fibrosus are made up of a strong protein called collagen which gets attached to the vertebrae above and below the IV disc. Likewise, the nucleus pulposus retains its jelly character because of the presence of large amounts of proteoglycans which are hydrophilic in character and hence have the ability to attract and absorb water molecules. This layer acts as the shock absorber to the load bearing vertebral column IV disc prolapse is a common condition wherein, a variety of genetic, environmental and acquired factors cause the outer annulus fibrosus to rupture leading to the prolapse of the inner nucleus pulposus which compresses the spinal cord and the spinal nerve roots which may lead to various neurological symptoms. Although this condition can occur at any vertebral level, it is most common in the lumbar area leading to pain, paresthesia, loss of muscle power in the lower limbs along with bladder and bowel control problems in some patients.

In the human body, there are 5 levels (i.e., 5 vertebral bones) of lumbar vertebrae. These 5 levels of the lumbar vertebrae are labeled as L1, L2, L3, L4, and L5. In the 5 levels of the lumbar vertebrae, there are intervertebral discs which are labeled as T12-L1, L1-L2, L2-L3, L3-L4, L4-L5 and L5-S1. For example, the disc between the lumbar vertebrae L4 and L5 is called L4-L5.

According to embodiments of present disclosure, for each lumbar intervertebral disc a risk-profile may be created. The creating module 116, of the system 102, may create the risk-profile corresponding to each lumbar intervertebral disc of the user 108. The risk-profile created may comprise annulus fibrosus related data and nucleus pulposusrelated data. Further, the annulus fibrosus related data and nucleus pulposusrelated data may be created in a following manner as shown in below tables.

TABLE 1
Annulus fibrosus related data

| | T12-L1 | L1-L2 | L2-L3 | L3-L4 | L4-L5 | L5-S1 |
|---|---|---|---|---|---|---|
| Percentage of lamellae that are | 23 | 31 | 22 | | | |
| incomplete (Desirable <50%) | | | | | | |
| Vertical angle between a vertebrae | | | 46 | 42 | 53 | |
| and the lamellae in an anterior | | | | | | |
| region (Desirable <65 degrees) | | | | | | |
| Vertical angle between the | | | 62 | 60 | 73 | |
| vertebrae and the lamellae in a | | | | | | |
| posterior region (Desirable <80 | | | | | | |
| degrees) | | | | | | |
| Percentage of fibers which cross | | | | 55 | 57 | 65 |
| obliquely in opposite directions | | | | | | |
| (Desirable >50%) | | | | | | |
| Degree of degeneration (Values | | | | | | |
| are age dependent) | | | | | | |
| Quantity of aggregate | | | | | | |
| proteoglycan (Values are age | | | | | | |
| dependent) | | | | | | |
| Keratinsulphate/chondroitin | | | | | | |
| sulphate ratio (Values are age | | | | | | |
| dependent) | | | | | | |

TABLE 2
Nucleus pulposus related data

| | T12-L1 | L1-L2 | L2-L3 | L3-L4 | L4-L5 | L5-S1 |
|---|---|---|---|---|---|---|
| Ratio between mucoidal material | | | | | | |
| to fibrocartilage (Values are age | | | | | | |
| dependent) | | | | | | |
| Number of discontinuities in | 0 | 0 | 0 | 1 | 0 | 2 |
| fibrocartilage (Desirable-0) | | | | | | |
| Number of discontinuities in | 0 | 0 | 1 | 1 | 0 | 2 |
| hyaline cartilage (Desirable-0) | | | | | | |
| Degree of degeneration (Values | | | | | | |
| are age dependent) | | | | | | |
| Quantity of aggregate | | | | | | |
| proteoglycan (Values are age | | | | | | |
| dependent) | | | | | | |
| keratin sulphate/chondroitin | | | | | | |
| sulphate ratio (Values are age | | | | | | |
| dependent) | | | | | | |

It may be observed from the above tables that the annulus fibrosus related data may comprises percentage of lamellae that are incomplete, vertical angle between a vertebrae and the lamellae in an anterior region, vertical angle between the vertebrae and the lamellae in a posterior region, percentage of fibers which cross obliquely in opposite directions, degree of degeneration, quantity of aggregate proteoglycan, and Keratinsulphate/chondroitin sulphate ratio. Further, the nucleus data may comprise ratio between mucoidal materials to fibrocartilage, number of discontinuities in fibrocartilage, number of discontinuities in hyaline cartilage, degree of degeneration, quantity of aggregate proteoglycan, keratin sulphate or chondroitin sulphate ratio.

According to embodiments of present disclosure, the risk-profile is generated using non-invasive imaging techniques. These techniques may comprise one or more computed tomography (CT), magnetic resonance imaging (MRI), magnetic resonance elastography (MRE), optical coherence tomography (OCT), and an ultrasound. Further, the risk-profile generated may be stored in the risk-profile database 126 of the system 102. Further, the risk-profile may be updated over a time interval based on the age and health of the user 108. For example, the risk-profile, for young and healthy users, may be updated at the time interval of one year, whereas, for old users, the risk-profile may be updated at the interval of every six months.

In the next step, the receiving module 118, of the system 102, may receive sensor-data corresponding to one or more muscle groups of user's interest and a lumbar region of the user 108. The lumbar region comprises 5 levels of the lumbar vertebrae and their adjoining IV discs. Further, the 5 levels of the lumbar vertebrae are labeled as L1, L2, L3, L4, and L5. Further, the sensor-data may indicate an effect of user's activity on the lumbar region of the user 108. Further, the sensor-data may be obtained from one or more sensors 130 comprising Electromyography sensors, 3-Dimensional accelerometers, 3-Dimensional gyroscopes, and compass. These one or more sensors 130 may be coupled with the wearable device 104 of the user 108. The one or more sensors 130 are capable of sensing or capturing different physical parameters of the user 108. The physical parameters are collectively referred as the sensor-data. According to embodiments of present disclosure, the sensor-data may comprise muscle tone data corresponding to the one or more muscle groups, information pertaining to a muscle activated during a physical activity, information pertaining to a muscle not activated during the physical activity, movements of the user 108, rate of change of movement, directional activity, hinged movement at hip of the user 108, twisting and movements of upper and lower limbs of the user 108. For example, the system 102 may compute a tension handled by the individual muscles based on the muscle tone data. Further, the sensor-data may be stored in the sensor database 128 of the system 102.

Further, the processing module 120, of the system 102, may process the risk-profile and the sensor-data based on a predefined value in order to determine information pertaining to a risk associated with the lumbar intervertebral disc prolapse. For example, the system 102 may monitor the instantaneous force acting on various IV discs and the maximum instantaneous force that can be safely handled by the IV disc at L3-L4 for a healthy 40 year old male with paraspinal muscle tone at the 50th percentile of the normalized population with no cartilaginous discontinuity could be set at 60N. If during any activity, such as lifting a heavy weight while bending forward with the upper body at a right angle to the lower limbs, the instantaneous force acting on the IV disc at L3-L4 may cross 60N. Then the system 102 will detect the activity as a risky one. The information pertaining to the risk may be further transmitted, by the transmitting module 122, to the wearable device 104 of the user 108. After receiving the information pertaining to the risk associated with the lumbar intervertebral disc prolapse, the wearable device 104 may generate alerts for the user 108. The alerts may be generated based on the postures or physical activities performed by the user 108. According to embodiments, an application may be installed in the wearable device 104. The application may enable the wearable device 102 to generated trend on the physical activity of the user 108 over a period of time. Further, the application may provide data about overworked and underworked muscles and also suggests relaxing techniques and exercises.

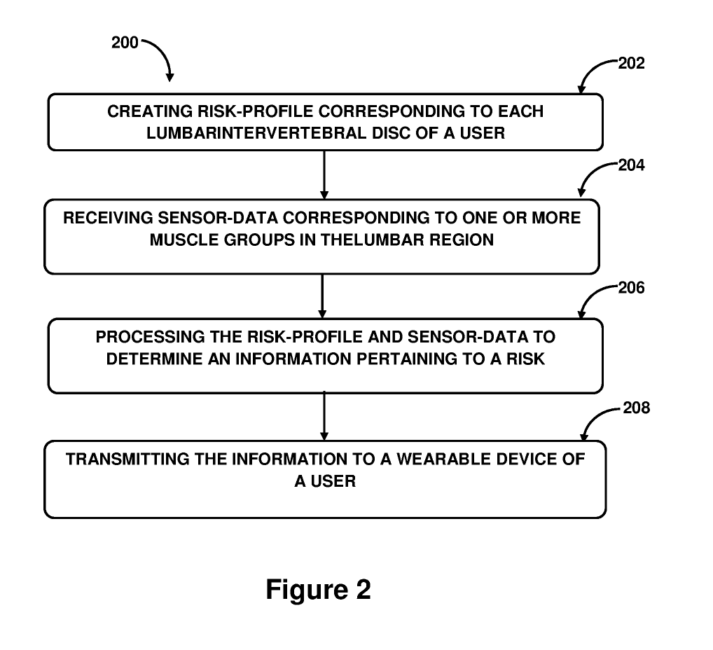

Referring now to FIG. 2, the method of determining risk associated with lumbar intervertebral disc prolapse is shown, in accordance with an embodiment of the present subject matter. The order in which the method 200 is described is not intended to be construed as a limitation, and any number of the described method blocks can be combined in any order to implement the method 200 or alternate methods. Additionally, individual blocks may be deleted from the method 200 without departing from the spirit and scope of the subject matter described herein. Furthermore, the method can be implemented in any suitable hardware, software, firmware, or combination thereof. However, for ease of explanation, in the embodiments described below, the method 200 may be considered to be implemented in the above described system 102.

At block 202, a risk-profile corresponding to each lumbar intervertebral disc may be created for the user. The risk-profile may comprise annulus fibrosus related data and nucleus pulposusrelated data.

At block 204, sensor-data corresponding to one or more muscle groups of user's interest and a lumbar region of the user may be received. The lumbar region may comprise L1 to L5 lumbar vertebrae and their adjoining IV discs. Further, the sensor-data may indicate an effect of user's activity on the lumbar region of the user.

At block 206, the risk-profile and the sensor-data may be processed based on a predefined value in order to determine information pertaining to a risk associated with the lumbar intervertebral disc prolapse.

At block 208, the information pertaining to the risk may be transmitted to the wearable device 104 of the user 108.

Although implementations for system and method for determining risk associated with lumbar intervertebral disc prolapse have been described in language specific to structural features and/or methods, it is to be understood that the appended claims are not necessarily limited to the specific features or methods described. Rather, the specific features and methods are disclosed as examples of implementations for determining risk associated with lumbar intervertebral disc prolapse.

We claim:

1. A method for determining risk associated with lumbar intervertebral disc prolapse, the method comprising:
Creating, by a processor, a risk-profile corresponding to each lumbar intervertebral disc of a user, wherein the risk-profile comprises annulus fibrosus related data and nucleus pulposus related data;
Receiving, by the processor, sensor-data corresponding to one or more muscle groups of user's interest and a lumbar region of the user, wherein the lumbar region comprises L1 to L5 lumbar vertebrae and their adjoining IV discs, and wherein the sensor-data indicates an effect of user's activity on the lumbar region of the user; and
Processing, by the processor, the risk-profile and the sensor-data based on a predefined value in order to determine information pertaining to a risk associated with the lumbar intervertebral disc prolapse.

2. The method of claim 1, wherein the risk-profile is generated using non-invasive imaging techniques comprising computed tomography (CT), magnetic resonance imaging (MRI), magnetic resonance elastography (MRE), optical coherence tomography (OCT), and an ultrasound.

3. The method of claim 1, wherein the sensor-data is obtained from sensors (130) comprising Electromyography sensors, 3-Dimensional accelerometers, 3-Dimensional gyroscopes, and compass.

4. The method of claim 1, further comprises transmitting the information pertaining to the risk to a wearable device (104) of the user, wherein the wearable device (104) generates an alert for the user based on the risk.

5. The method of claim 1, wherein the annulus fibrosus related data comprises percentage of lamellae that are incomplete, vertical angle between a vertebrae and the lamellae in an anterior region, vertical angle between the vertebrae and the lamellae in a posterior region, percentage of fibers which cross obliquely in opposite directions, degree of degeneration, quantity of aggregate proteoglycan, and Keratinsulphate/chondroitin sulphate ratio.

6. The method of claim 1, wherein the nucleus pulposusrelated data comprises ratio between mucoidal material to fibrocartilage, number of discontinuities in fibrocartilage, number of discontinuities in hyaline cartilage, degree of degeneration, quantity of aggregate proteoglycan, keratin sulphate or chondroitin sulphate ratio.

7. The method of claim 1, wherein the sensor-data comprises muscle tone data corresponding to the one or more muscle groups, information pertaining to a muscle activated during a physical activity, information pertaining to a muscle not activated during the physical activity, movements of the user, rate of change of movement, directional activity, hinged movement at hip of the user, twisting and movements of upper and lower limbs of the user.

8. A system (102) for determining risk associated with lumbar intervertebral disc prolapse, wherein the system (102) comprises:
a processor (110); and
a memory (114) coupled to the processor (110), wherein the memory (114) has a plurality of modules stored therein that are executable by the processor (110), the plurality of modules comprising:
a creating module 116 to create a risk-profile corresponding to each lumbar intervertebral disc of a user, wherein the risk-profile comprises annulus fibrosus related data and nucleus pulposus related data;
a receiving module 118 to receive sensor-data corresponding to one or more muscle groups of user's interest and a lumbar region of the user, wherein the lumbar region comprises L1 to L5 lumbar vertebrae and their adjoining IV discs, and wherein the sensor-data indicates an effect of user's activity on the lumbar region of the user; and
a processing module 120 to process the risk-profile and the sensor-data based on a predefined value in order to determine information pertaining to a risk associated with the lumbar intervertebral disc prolapse.

9. The system (102) of claim 8, wherein the risk-profile is generated using non-invasive imaging techniques comprising computed tomography (CT), magnetic resonance imaging (MRI), magnetic resonance elastography (MRE), optical coherence tomography (OCT), and an ultrasound.

10. The system (102) of claim 8, wherein the sensor-data is obtained from sensors (130) comprising Electromyography sensors, 3-Dimensional accelerometers, 3-Dimensional gyroscopes, and compass.

11. The system (102) of claim 8, further comprising a transmitting module (122) to transmit the information pertaining to the risk to a wearable device (104) of the user, wherein the wearable device (104) generates an alert for the user based on the risk.

12. A non-transitory computer readable medium embodying a program executable in a computing device for determining risk associated with lumbar intervertebral disc prolapse, the program comprising:
a program code for creating risk-profile corresponding to each lumbar intervertebral disc of a user, wherein the risk-profile comprises annulus fibrosus related data and nucleus pulposus related data;
a program code for receiving sensor-data corresponding to one or more muscle groups of user's interest and a lumbar region of the user, wherein the lumbar region comprises L1 to L5 lumbar vertebrae and their adjoining IV discs, and wherein the sensor-data indicates an effect of user's activity on the lumbar region of the user; and
a program code for processing the risk-profile and the sensor-data based on a predefined value in order to determine information pertaining to a risk associated with the lumbar intervertebral disc prolapse.
